(19) United States
(12) Patent Application Publication
(10) Pub. No.: US 2010/0049287 A1
(43) Pub. Date: Feb. 25, 2010

(54) Porous medical dorsal column self anchoring lead

(75) Inventor: Anthony Alexander, Seymour, IN (US)

Correspondence Address: BRYAN CAVE LLP, 211 NORTH BROADWAY, SUITE 3600, ST. LOUIS, MO, 63102-2750, US

(73) Assignee: Alexander Medical Anchors, Inc., Seymour, IN (US)

(21) Appl. No.: 12/386,824

(22) Filed: Apr. 23, 2009

Related U.S. Application Data
(60) Provisional application No. 61/047,330, filed on Apr. 23, 2008.

(51) Int. Cl. A61N 1/05

(52) U.S. Cl. 607/116

(57) ABSTRACT

Implantable leads for use in, inter alia, neurostimulation therapy and that comprise a coagulant reservoir accessible by a coagulant access port are disclosed. The coagulant reservoir comprises a porous membrane. When such a lead is implanted into a patient, coagulant fluid (preferably blood plasma) is injected or otherwise inserted into the coagulant reservoir via the coagulant access port. Coagulant fluid can then flow out of the coagulant reservoir, via coagulant apertures in the porous membrane, into the patient's body. Systems comprising such leads are also disclosed, as are methods of manufacturing such systems and/or leads.

### RELATED APPLICATION INFORMATION

This application claims the benefit of U.S. Provisional Patent Application Ser. No. 61/047,330, filed on Apr. 23, 2008, the disclosure of which is incorporated herein by reference in its entirety for all purposes.

### FIELD OF THE INVENTION

This invention relates to implantable medical devices and, more particularly, implantable medical leads.

### BACKGROUND OF THE INVENTION

Neurostimulation therapy is used to treat a variety of symptoms or conditions such as chronic pain, tremor, Parkinson's disease, multiple sclerosis, spinal cord injury, cerebral palsy, amyotrophic lateral sclerosis, dystonia, epilepsy, post-laminectomy syndrome (also known as failed back syndrome), urinary incontinence, fecal incontinence, sexual dysfunction, obesity, or gastroparesis or other gastric mobility disorders.

Neurostimulation therapy can be delivered to a patient using a neurostimulation system comprising one or more implantable leads. A lead will comprise a plurality of proximal contact points capable of electrical communication with a plurality of distal contact points, via a conductive material (e.g., a conductive wire). A neurostimulation system will further comprise at least one source for generating an electrical current, e.g., a generator comprising at least one battery, wherein the electrical source is capable of being electrically coupled with the proximal contact points of a lead. Distal contact points can be placed on, near, or otherwise in communication with a portion of a patient, e.g., a portion of a patient expected to benefit from neurostimulation therapy such as the spinal cord, pelvic nerves, stomach, or gastrointestinal tract, or within the cranium of a patient, e.g., for deep brain stimulation or occipital nerve stimulation.

Accurate targeting of the electrical stimulation of these systems is very important to treatment efficacy, as is maintenance of the position of the lead within the patient, relative to the treatment site. Accordingly, a critical component to the effectiveness these systems is secure and stable anchoring of leads used to deliver treatment. Leads used in connection with current neurostimulation systems are prone to displacement following implantation. A displaced lead can minimize or eliminate the effectiveness of neurostimulation therapy and may require further surgery to correct. A lead less prone to movement away from the treatment zone following implantation could enhance the effectiveness of neurostimulation therapy and minimize the need for future corrective procedures. Accordingly, a need exists for leads that are less prone to displacement.

### BRIEF SUMMARY OF THE INVENTION

An invention having various embodiments that meets this need has now been developed. In general, a lead of this invention comprises a porous membrane near (and preferably proximal to) the distal contacts of the leads, wherein the porous membrane defines a coagulant reservoir, wherein the coagulant reservoir is adapted to receive a coagulant fluid, which coagulant fluid can then pass through the porous membrane into a patient. The coagulant fluid will then encourage adhesion of the lead, at the porous membrane, to the patient's tissues.

A preferred coagulant is blood plasma; particularly preferred is blood plasma of the patient in whom a lead of this invention is being implanted. Selected components of blood plasma (e.g., coagulation factors of blood plasma) and/or other naturally occurring and/or synthetic coagulants may be used as (or as a component of) the coagulant fluid with leads of this invention. Although the inventor does not wish to be constrained by any hypothesis, it is believed that when the coagulant fluid used with a lead of this invention comprises blood plasma, the fibrin, cross-linked fibrin clot, and/or other coagulation factors present in the blood plasma and/or created during the coagulation cascade act as an adhesive connecting the lead to the patient's tissue, which then promote the formation of “scar tissue” and a more permanent connection. Thus, the lead becomes “self-anchoring”, in that it promotes a connection between the lead and the patient's tissues.

These and other aspects and embodiments of this invention are more fully described below.

### BRIEF DESCRIPTION OF THE DRAWINGS

The invention will be better understood from a reading of the following detailed description, taken in conjunction with the accompanying Figures in the drawings in which:

FIG. 1 is an isometric view of a neurostimulation system according to one embodiment of the invention;

FIG. 2 is a closer view of the neurostimulation system of FIG. 1;

FIG. 3 is an isometric view of a neurostimulation system according to a second embodiment of the invention;

FIG. 4 is a side view of a lead according to an embodiment of the invention;

FIG. 4A is a close up view of a portion of the porous membrane of the lead of FIG. 4A;

FIG. 4B is a close up view of a portion of the distal contacts of the lead of FIG. 4;

FIG. 5 is a top isometric view of a porous membrane according to an embodiment of the invention, wherein the porous membrane is not attached to a lead;

FIG. 6 is a left, isometric cut-away view of a coagulant reservoir of a lead according to an embodiment of the invention;

FIG. 7 is a left, isometric cut-away view of a coagulant reservoir of a lead according to an embodiment of the invention;

FIG. 8 is a right, isometric view of a portion of a lead according to an embodiment of the invention;

FIG. 9 illustrates implanted leads of a neurostimulation system according to an embodiment of the invention;

FIG. 10 is a close-up view of an implanted leads of a neurostimulation system according to an embodiment of the invention;

FIG. 11 is a left, front isometric view of a neurostimulation system according to an embodiment of the invention;

FIG. 12 is a left, front isometric view of a neurostimulation system according to an embodiment of the invention; and

FIGS. 13-17 illustrate assembly of a lead of this invention according to a method of this invention.

For simplicity and clarity of illustration, the figures illustrate the general manner of construction; descriptions and details of well-known features and techniques may be omitted to avoid unnecessarily obscuring the invention. Additionally, elements in the figures are not necessarily drawn to scale. The same reference numerals in different figures denote the same elements.

The terms “first,” “second,” and the like in the description and in the claims, if any, are used for distinguishing between similar elements and not necessarily for describing a particular sequential or chronological order. It is to be understood that the terms so used are interchangeable under appropriate circumstances such that the embodiments of the invention described herein are, for example, capable of operation in sequences other than those illustrated or otherwise described herein. Furthermore, the terms “include,” “have,” and any variations thereof, are intended to cover a non-exclusive inclusion, such that a process, method, article, or apparatus that comprises a list of elements is not necessarily limited to those elements, but may include other elements not expressly listed or inherent to such process, method, article, or apparatus.

The terms “top,” “bottom,” “over,” “under,” “proximal,” “distal,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein.

The term “coupled,” as used herein, is defined as directly or indirectly connected in an electrical, mechanical, or other manner. For example, a first mechanism electrically coupled to a second mechanism may be directly connected or may be indirectly connected via a conductor coupled to the first and second mechanisms that operates to transmit signals from the first mechanism to the second mechanism. The term “attached,” as used herein, is defined as firmly securing, joining, fixing, fastening, or otherwise connecting one item to another item (in a removable or non-removable) manner, either directly or via one or more intermediate items secured to the items described as “attached” to one another in a manner appropriate for the specific items.

### DETAILED DESCRIPTION OF THE INVENTION

Numerous implantable leads and neurostimulation systems comprising implantable leads have been developed for neurostimulation therapy and/or other types of therapy, treatment, and/or monitoring, the general purpose of which is to deliver electrical current from a generator to the areas within a patient targeted for therapy, treatment, and/or monitoring. By way of example only, U.S. Pat. No. 7,519,432, incorporated herein by reference, describes several leads.

Typically, a lead for use in neurostimulation therapy (or other electrical stimulation therapy) will comprise a plurality of proximal contact points each of which is electrically coupled to one of a plurality of distal contact points via a conductor; distal contact points typically are implanted in contact with a patient's tissue, e.g., in the spinal column, and are used to deliver therapy and/or monitor physiological conditions of a patent, or both. A typical conductor is a wire (or plurality of wires) contained within the lead that spans the distance between a proximal contact point and a distal contact point. Leads may further comprise elements such as a stylet guide.

Commonly, a lead will comprise four or eight proximal and four or eight distal contact points and a conductor will electrically connect a single proximal contact point with a single distal contact point; other numbers of contact points and configurations are possible.

As noted above, the electrical pulses of neurostimulation therapy (or other electrical stimulation therapy or monitoring, e.g., cardiac or cochlear) are typically delivered to a patient via the distal contact points of a lead, which are placed in contact with the patient's tissue. As used herein, the “distal contact portion” of a lead refers to the portion of the lead that comprises one or more distal contact points; the “proximal contact portion” of a lead refers to the portion of the lead that comprises one or more proximal contact points.

In addition to the conductive portions of a lead, a lead will typically comprise non-conductive portions, such as spacers between contact points and an insulative sheath that surrounds the conductors. A low durometer plastic or polymer, such as polypropylene, polyethylene, polyurethane, silicone, or an alloy of one or more such plastic or polymer, may be used for the insulative sheath.

As described in further detail herein, this invention provides improved implantable leads, improved neurostimulation systems comprising implantable leads, and porous membranes capable of being attached to a lead to create a coagulant reservoir (and a lead and/or neurostimulation system of this invention). In addition to one or more of the components, features, and/or aspects of leads, neurostimulation systems, and porous membranes of this invention described herein, leads of this invention and/or neurostimulation systems comprising leads of this invention may comprise other components, features, and/or aspects of leads, including but not limited to those described above.

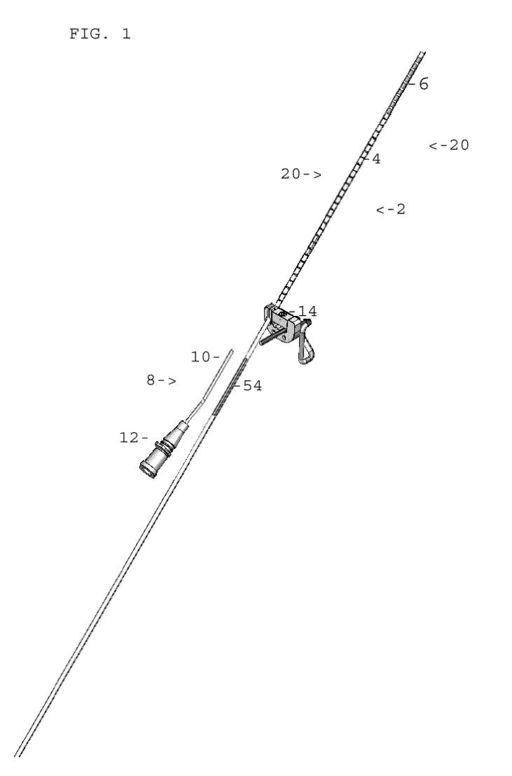

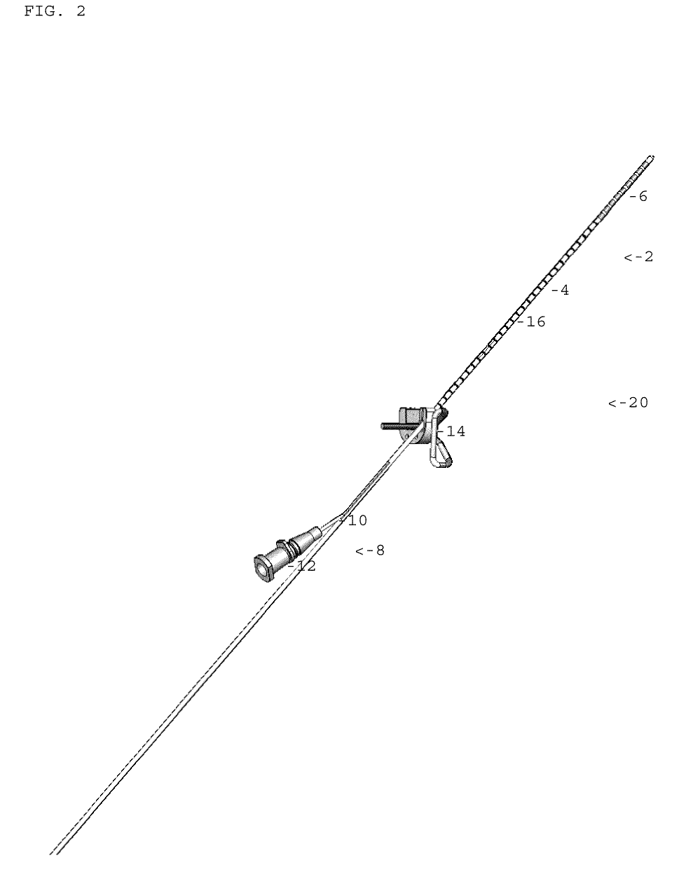

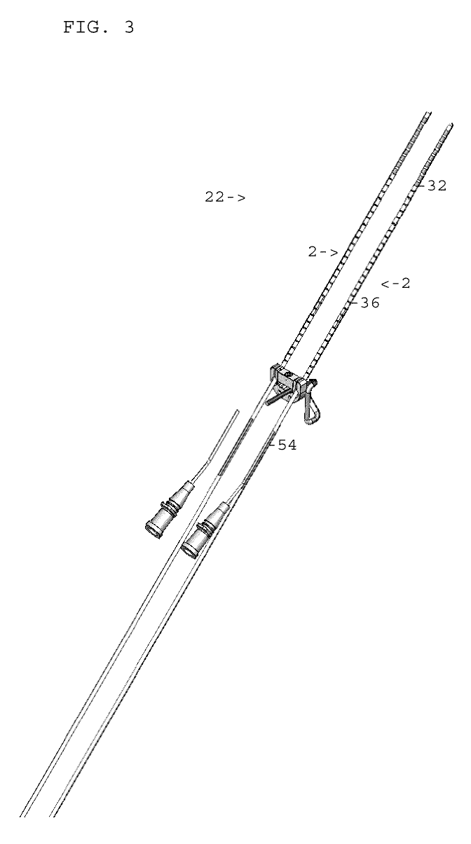

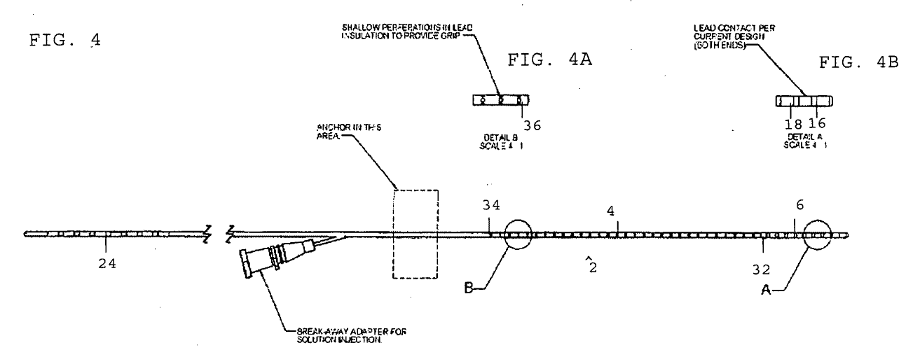

Embodiments of leads 2 of this invention and of neurostimulation systems 20, 22 of this invention are illustrated, inter alia, in FIG. 1-4B. In the illustrated embodiment, the lead 2 comprises a distal contact portion 6 and a proximal contact portion 24, each of which comprises a plurality of contacts 16 and spacers 18. The proximal contact portion 24 is capable of being electrically coupled with a generator (not shown).

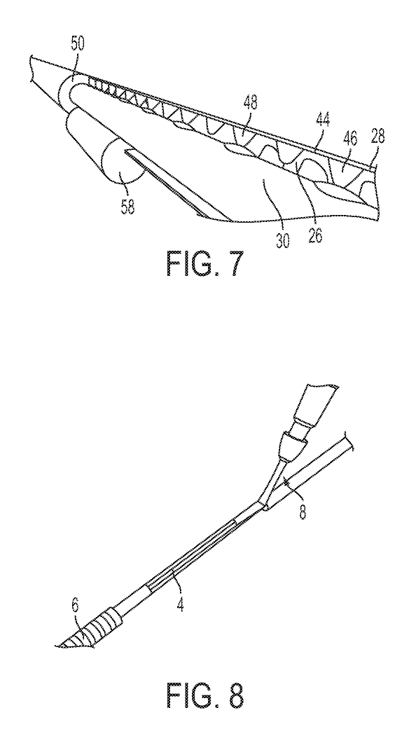

As shown in the cross-section illustrations of FIG. 6 and 7, a lead of this invention comprises a coagulant reservoir 26 near the distal contact portion 6 of the lead 2. A coagulant reservoir 26 may comprise an outer wall 28, an inner wall 30, a first end 32, and a second end 34. The outer wall 28 of a coagulant reservoir 26 comprises a porous membrane 4. Axially, a coagulant reservoir 26 will preferably span the entire circumference of the lead. However, a coagulant reservoir that spans less than the entire circumference of the lead may be used in a lead of this invention. Longitudinally, the a coagulant reservoir 26 is preferably between approximately 16 centimeters and approximately 25 centimeters in length. The length of a coagulant reservoir 26 need not be the same for the entire distance of its axial span. Thus, the “length of the coagulant reservoir” refers to its length along some portion of the circumference of the lead, i.e., between the first end and the second end of the coagulant reservoir. More than one coagulant reservoir may be provided in a lead of this invention.

A porous membrane 4 of a lead of this invention may be made of a low durometer plastic or polymer, such as polypropylene, polyethylene, polyurethane, silicone, or an alloy of one or more such plastic or polymer. Preferably, a porous membrane is made of the same or substantially the same material as the insulative sheath of the lead comprising the porous membrane. Ideally, the material used to make porous membrane of this invention will not notably increase the tensile strength or stiffness of the lead to which it is applied.

The porous membrane 4 will form an outer wall 28 around all or a portion of the circumference of the lead along a portion of the length of the lead. In the illustrated embodiments, the porous membrane 4 surrounds the entire circumference of the lead and extends, in length, from the proximal end of the distal contact portion 6 of the lead 2 toward the proximal contact portion 24 of the lead 2. Again, a preferred length for the porous membrane is between approximately 16 centimeters and approximately 25 centimeters in length.

In one embodiment, the distance (along the length of the lead) between the distal end of the proximal contact portion and the second end 34 of the coagulant reservoir is between approximately 16 centimeters and approximately 25 centimeters in length, the distance (along the length of the lead) between the second end 34 of the coagulant reservoir and the first end 32 of the coagulant reservoir is between approximately 16 centimeters and approximately 25 centimeters, and the first end of the coagulant reservoir at or very near the proximal end of the distal contact portion.

The porous membrane 4 of a coagulant reservoir 26 will comprise a plurality of pores, holes, or other apertures through which a coagulant fluid in the coagulant reservoir 26 can flow or otherwise pass (a “coagulant aperture”). It is generally preferred that a coagulant aperture comprises a cavity in which such cavity further comprises an interior aperture (an aperture open to the interior of the coagulant reservoir), an exterior aperture (an aperture open to the exterior surface of the porous membrane), and a hollow body connecting the interior aperture to the exterior aperture, wherein the hollow body is contained (or substantially contained) within the coagulant reservoir (a “protruding coagulant aperture”). Particularly preferred is a protruding coagulant aperture in which the interior aperture is smaller than the exterior aperture. Although not bound to this theory, the inventor believes such a protruding coagulant aperture is particularly suited for facilitating connection of the lead at the porous membrane to the patient's tissue in a manner that will provide a reasonably firm adhesion to the patient's tissue, but will allow the lead to be subsequently removed (if necessary or otherwise desirable) with minimal damage to the underlying tissue.

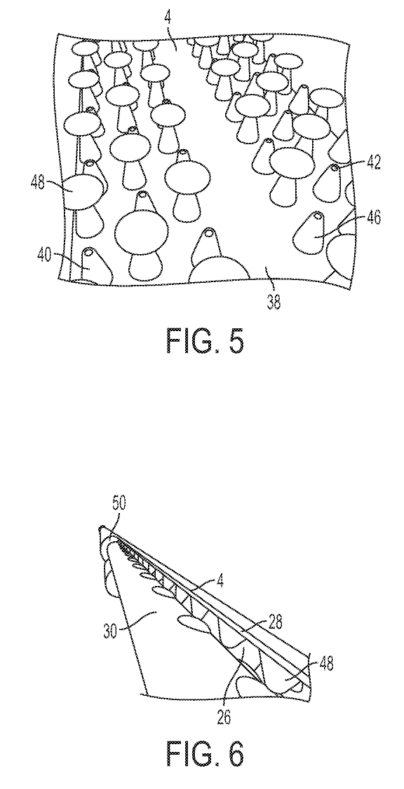

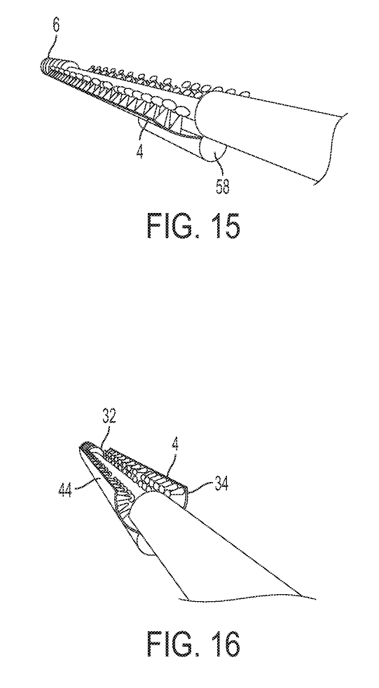

In a preferred embodiment, as illustrated on FIGS. 5-7 and 13-16, the interior surface 38 of the porous membrane 4 comprises a plurality of conically-shaped protruding coagulant apertures 40 wherein the conically-shaped protruding coagulant apertures 40 are open to both the interior of the coagulant reservoir 26 at an interior aperture 42 (e.g., via a hole or other aperture at or near the top of the conically-shaped protruding coagulant aperture 40) and to the external surface 44 of the porous membrane 4 at an external aperture 36, wherein the apertures are connected by the hollow body 46 formed by the conically-shaped protruding coagulant aperture. In one embodiment, the diameter of the interior aperture 42 of a conically-shaped protruding coagulant aperture 40 is approximately one-half of the diameter of the exterior aperture 36. Optionally, the height of a conically-shaped protruding coagulant aperture 40 (or other protruding coagulant aperture) may be in the range of approximately 0.5 millimeters to 1.5 millimeters.

Although protruding coagulant apertures are preferred, all or a portion of the coagulant apertures may comprise a hole or other opening in the porous membrane. The porous membrane may comprise a mesh in which coagulant apertures comprise the open portions (e.g., interstices) of the mesh.

A lead of this invention may optionally include one or more supporting members 48 disposed within a coagulant reservoir that extend between the inner wall 30 and the outer wall 28 of a coagulant reservoir 26. Preferably, the height of a supporting member 48 is somewhat greater than the height of the protruding coagulant apertures (if any); in such an embodiment wherein distance between the interior surface of the porous membrane 36 and the inner wall 30 of the coagulant reservoir 26 is substantially the same as the height of the supporting member, some space remains between the interior aperture 42 of the protruding coagulant aperture 40 and the inner wall 30.

A supporting member, for example, may be a columnar-shaped member. A supporting member may optionally take the form of a ring around the inner wall of the coagulant reservoir and in contact with the inner surface of the porous membrane; in such an embodiment, a supporting member would preferably include one or more holes or other apertures to allow the passage of coagulant fluid to any portion of the coagulant reservoir beyond such a supporting member. A supporting member made of the same material as the porous membrane and/or the inner wall (and/or formed as an integral part of either and/or both the inner wall and the porous membrane) or a different but suitable material; other shapes may be used for a supporting member In an embodiment of a lead and/or neurostimulation system of this invention comprising more than one supporting member, the supporting members may be provided in a variety of shapes and configurations.

Optionally, the height of a supporting member may be in the range of approximately 0.5 millimeters to 2.0 millimeters. For example, in an embodiment of a lead, system, and/or porous membrane comprising both a protruding coagulant aperture and a supporting member, the height of the supporting member may be about double the height of the protruding coagulant aperture. Generally, interior aperture diameters, exterior aperture diameters, protruding coagulant aperture heights, and/or supporting member heights selected for leads, systems, and/or porous membranes of the invention may be based at least in part on the viscosity of the coagulant fluid that will be contained within the coagulant reservoir in a particular embodiment of a lead and/or system of the invention.

FIGS. 5-7 and 13-16 illustrate a preferred embodiment of a supporting member; such supporting members 48 protrude from the interior surface 38 of the porous membrane 4. As shown on the figures, in this embodiment, the supporting members 48 are mushroom shaped. As noted above, however, other shapes may be used for supporting members and/or supporting members of a particular may be provided in a variety of shapes.

The invention further provides porous membranes 4 capable of being attached to a lead to form a coagulant reservoir beneath some portion or all of the porous membrane. Such a porous membrane may comprise one or more of the features described above; specifically, a plurality of coagulant apertures, one or more supporting members, and/or a coagulant access port.

As will be appreciated by those with skill in the art, the porous membrane at the points that do not include an optional protrusion will be very thin.

The inner wall 30 of a coagulant reservoir 26 is preferably made of a low durometer plastic or polymer, such as polypropylene, polyethylene, polyurethane, silicone, or an alloy of one or more such plastic or polymer. A preferred inner wall 30 of a coagulant reservoir 26 is the portion of the insulative sheath of the lead that lies below the porous membrane composing the coagulant reservoir. Such an embodiment is illustrated in FIGS. 6, 7, and 13-16. Generally, though, a inner wall of a coagulant reservoir will lie substantially opposite (or beneath) the porous membrane and will be sufficient less porous than the porous membrane such that coagulant fluid within the coagulant reservoir would be more likely to flow out of the coagulant reservoir through the coagulant apertures and into the patient's body than through the inner wall toward the interior of the lead.

The first 32 and second 34 ends of the coagulant reservoir 26 refer generally to the points along the lead that mark the proximal and distal boundaries of the coagulant reservoir 26. The first and/or second end may comprise a wall or wall-like structure, e.g., a portion of the insulative sheath of the lead with a circumference greater than the circumference of the inner wall of the coagulant reservoir 50, 52. In this embodiment, as illustrated inter alia on FIGS. 6, 7, 13, and 14, the circumference of the lead at the coagulant reservoir may be substantially the same as the circumference of the lead on either side of the coagulant reservoir. The first and/or second end of a porous membrane 4 of this invention could be attached (e.g., clamped, glued, taped, adhered, or otherwise secured) around what would otherwise be the external surface of the lead to form a coagulant reservoir in which the distal and proximal sites of attachment of the porous membrane 4 could comprise the first and/or second end of the coagulant reservoir.

A lead of the present invention could further comprise a coagulant access port 54 that is (or is capable of becoming) in fluid communication with a coagulant reservoir 26 and is adapted to receive an injection and/or infusion of coagulant.

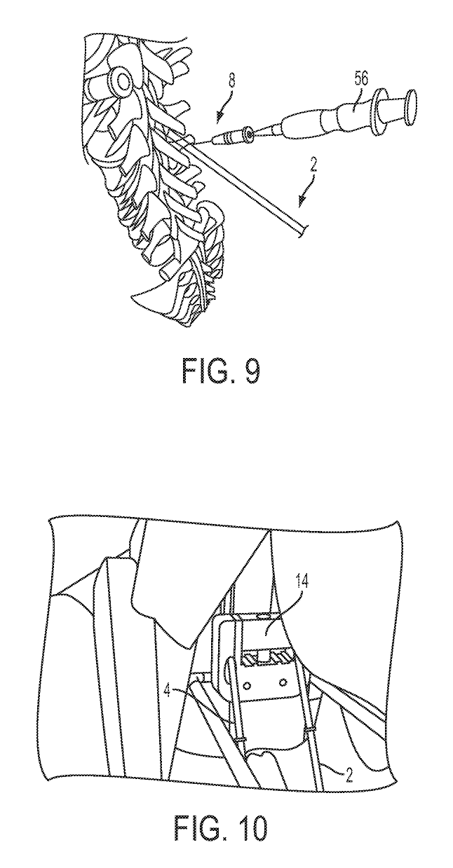

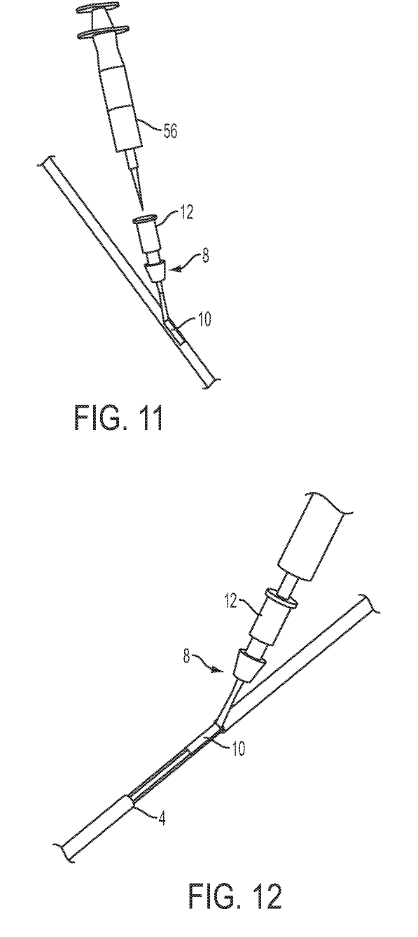

A lead may optionally comprise an access port coupling device, e.g., a device adapted to allow and/or otherwise facilitate the flow of coagulant fluid from a coagulant fluid housing (e.g., a syringe containing coagulant fluid) into the coagulant fluid reservoir 26 via the coagulant access port 54. For example, the embodiment illustrated, inter alia, on FIGS. 1, 2, 9, 11, 12, and 19 comprises an access port coupling device 8; the access port coupling device 8 comprises a cannula 10 (such as the type of cannula typically used in connection with administering drugs or fluids intravenously) coupled to an injection port 12. In such an embodiment, coagulant fluid from a syringe 56 (or other coagulant fluid housing) could be injected into the access port coupling device 8 and coagulant fluid could then flow through the coagulant access port 34, into the coagulant reservoir 26, and out the coagulant apertures of the porous membrane 4.

A lead of the present invention may be prepared for insertion into a patient with an access port coupling device 8 attached or otherwise coupled to the lead at the coagulant access port 34, e.g., via tape, surgical glue, a plastic membrane, or other suitable adhesive. All or a portion of the access port coupling device (e.g., a cannula or a cannula and an injection port) could be formed as an integral part of the lead, heat sealed to the lead, and/or otherwise attached to the lead.

Alternatively, the lead could be adapted for use with an access port coupling device 8 but prepared for insertion into a patient without the access port coupling device 8 attached or otherwise coupled to the lead 2; the access port coupling device 8 could be inserted into, attached, and/or otherwise coupled with the coagulant access port 34 as a step in the implantation procedure. In such an embodiment, the lead may optionally include a coagulant access port plug 58 that is removably attached to the lead 2 at the coagulant access port 34. The coagulant access port plug 58 could be removed to provide access to the coagulant access port, e.g., for inserting or otherwise coupling an access port coupling device 8.

Unless specifically stated otherwise, references to a neurostimulation system comprising a lead and an access port coupling device shall refer to embodiments in which a lead is provided with an access port coupling device attached, secured, and/or otherwise coupled to the lead as well as to embodiments in which an access port coupling device is subsequently attached, secured, and/or otherwise coupled, by the manufacturer, a third party, a physician, or otherwise, to the lead (such as, for example, to embodiments in which an access port coupling device is coupled to the lead by a physician during an implantation procedure)

Other forms of coagulant access port may be used. For example, coagulant fluid could be injected though the porous membrane into the coagulant reservoir, e.g., via a cannula coupled to a syringe adapted to puncture the porous membrane. In such an embodiment, the term “coagulant access port” (or coagulant access ports) would simply refer to the point (or points) at which the coagulant fluid is (or could be) injected into the coagulant reservoir.

The coagulant access port may represent a point at which a lumen or tube enters the coagulant reservoir. Such a lumen or tube may pass through a portion of the lead and be in fluid communication with a coagulant fluid housing. Generally, a coagulant access port refers to a point at which coagulant fluid is or can be, directly or indirectly, injected, infused, or otherwise placed into a coagulant reservoir.

A preferred coagulant is blood plasma; particularly preferred is blood plasma of the patient in whom a lead of this invention is being implanted. Selected components of blood plasma (e.g., coagulation factors of blood plasma) and/or other naturally occurring and/or synthetic coagulants may be used as (or as a component of) the coagulant fluid with leads of this invention. Although the inventor does not wish to be constrained by any hypothesis, it is believed that when the coagulant fluid used with a lead of this invention comprises blood plasma, the fibrin, cross-linked fibrin clot, and/or other coagulation factors present in the blood plasma and/or created during the coagulation cascade act as an adhesive connecting the lead to the patient's tissue, which then promote the formation of “scar tissue” and a more permanent connection. Thus, the inventor believes the lead will become “self anchoring” and will be particularly suited to resist horizontal movement.

A lead 2 comprising a coagulant reservoir 26 may be coupled to an anchor 14, preferably at or near the coagulant reservoir 26 (as show in FIGS. 1-3 and 10) to compose an embodiment of a neurostimulation system of this invention. Preferred anchors for use in such a system are anchors described and disclosed in U.S. patent application Ser. No. 12/412,275 filed Mar. 26, 2009, incorporated herein by reference in its entirety. The inventor believes such a system will minimize longitudinal movement (accomplished predominately by the anchor) and lateral movement (accomplished predominately by the “self-anchoring” aspects of the lead of the present invention), thus providing an improved lead resistant to displacement following implantation.

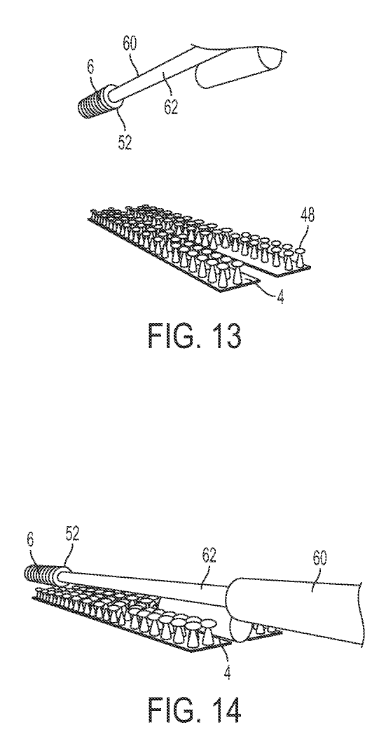

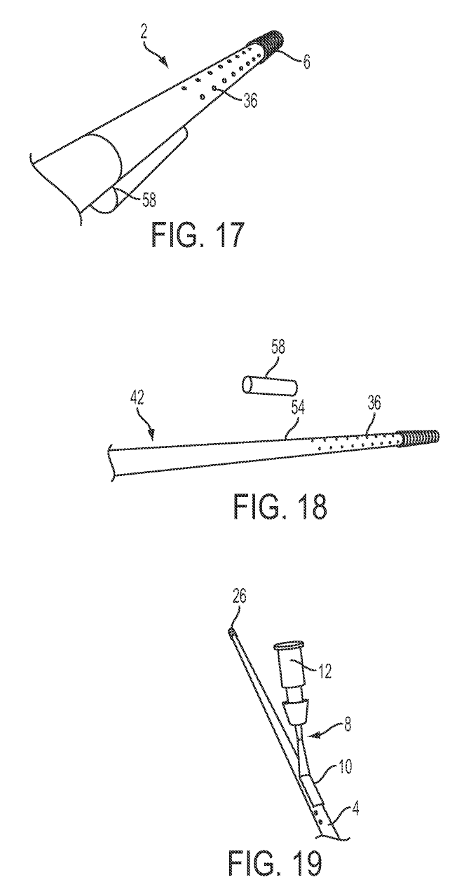

Although leads and neurostimulation systems of the present invention are not limited to any particular method of manufacture, this invention includes methods of manufacture that may be used to create a porous membrane of this invention, a lead of this invention, and/or neurostimulation system comprising a lead of this invention. For example, such a method of manufacture of this invention may comprise one or more of the following steps, illustrated in part in FIGS. 13-19:
A porous membrane 4 is created according to one or more of the following steps: (1) a suitable plastic or polymer (e.g., polypropylene) is provided in resin form, (2) the resin is heated (i.e., melted) and rolled into a sheet that is the desired thickness of the porous membrane, (3) a punch creates protrusions in the sheet, e.g., mushroom shaped protrusions, and (4) tops (e.g., the “top” of the “mushroom”) are cut or otherwise removed from a portion of the protrusions; in one embodiment, tops are removed from about ⅔^{rd} of the protrusions.
A lead body 60, is provided, wherein “lead body” refers to an implantable lead comprising proximal contact points electrically coupled (or capable of being electrically coupled) to distal contact points via one or more conductors, and, optionally, such other components, features, and/or aspects as may be necessary or desirable given the intended and/or anticipated use of the lead. Optionally, the lead body 60 is adapted to receive the porous membrane 4 by one or more of following: (1) an access port plug 58 is provided on the lead body 60, and/or (2) the lead body 60 comprises a radius at the site of the expected application point 62 of the porous membrane (e.g., at the site of the expected coagulant reservoir) that is smaller than the radius of the lead on either end of the site of the expected application point 62 of the porous membrane; ideally, the difference in radius will be approximately equal to the height of the protruding support members 48 of the porous membrane 4.
The porous membrane 4 is wrapped around the lead body 60 and heat sealed or otherwise affixed, attached, and/or otherwise coupled to the lead body, thus creating a lead 2 according to an embodiment of this invention.
To create a neurostimulation system of this invention comprising a lead and an access port coupling device 8, the access port plug 58 (if provided) may be removed (see FIG. 18) and an access port coupling device 8 may affixed, attached, and/or otherwise coupled to the lead at the coagulant access port 54. Preferably, the access port coupling device is attached in a manner that promotes ease of removal of the access port coupling device after it has been used in connection with the transfer of coagulant fluid into the coagulant reservoir.

In general, leads and/or neurostimulation systems of the present invention can be implanted and/or used in conjunction with neurostimulation techniques currently known or subsequently developed. However, methods of implanting a lead of the present invention will comprise the further step of providing coagulant fluid to the coagulant reservoir 26 via a coagulant access port 54. If an optional access port coupling device is provided, it is preferably removed after the coagulant fluid has been injected or otherwise provided.

If an anchor will be used with a lead of the present invention, it is preferably installed after the coagulant fluid has been injected or otherwise provided.

Although the inventions disclosed herein have been primarily described in terms of neurostimulation therapy, leads and systems of this invention may be used in other therapeutic techniques comprising the use of an implantable lead. Furthermore, this invention is further directed to other implantable medical devices comprising a coagulant reservoir and a porous membrane and methods of using such implantable medical devices comprising providing blood plasma into the coagulant reservoir, which blood plasma can then pass into the patient's body to promote adhesion (e.g., via the formation of scar tissue) to the patient's tissues.

A “patient” may be a human or non-human animal patient.

Although certain illustrative embodiments have been disclosed, it will be apparent from the foregoing disclosure to those skilled in the art that variations and modifications of such embodiments may be made without departing from the spirit and scope of the invention. Accordingly, it is intended that the invention should be limited only to extent required by the appended claims and the rules and principals of applicable law.

I claim:

1. An implantable lead comprising a coagulant reservoir, wherein the coagulant reservoir comprises a porous membrane and a coagulant access port and wherein the porous membrane comprises a plurality of coagulant apertures.

2. The lead of claim 1 wherein the coagulant apertures are protruding coagulant apertures.

3. The lead of claim 2 wherein the protruding coagulant apertures are conically-shaped protruding apertures.

4. The lead of claim 3 wherein each conically-shaped protruding aperture comprises (a) an interior aperture comprising an interior aperture diameter, and (b) an exterior aperture comprising an exterior aperture diameter, and wherein the interior aperture diameter is smaller than the exterior aperture diameter.

5. The lead of claim 4 wherein the exterior aperture diameter is approximately twice as big as the interior aperture diameter.

6. The lead of claim 2 further comprising a supporting member.

7. The lead of claim 3 further comprising a supporting member.

8. The lead of claim 7 in which the supporting member is mushroom-shaped.

9. The lead of claim 6 in which each protruding coagulant aperture comprises an aperture height is between approximately 0.5 millimeters and approximately 0.75 millimeters and in which the supporting member comprises a member height that is greater than the aperture height.

10. The lead of claim 7 in which each protruding coagulant aperture comprises an aperture height is between approximately 0.5 millimeters and approximately 0.75 millimeters and in which the supporting member comprises a member height that is greater than the aperture height.

11. The lead of claim 1 further comprising coagulant fluid within the coagulant reservoir.

12. The lead of claim 11 wherein the coagulant fluid comprises blood plasma.

13. A neurostimulation system comprising:
a lead, wherein the lead comprises a coagulant reservoir, wherein the coagulant reservoir comprises a coagulant access port and a plurality of coagulant apertures; and
an access port coupling device.

14. The system of claim 13 further comprising a coagulant fluid housing wherein the coagulant fluid housing comprises coagulant fluid.

15. The system of claim 14 wherein the coagulant fluid comprises blood plasma.

16. The system of claim 11 further comprising a plurality of protruding coagulant apertures and a plurality of supporting members.

17. The system of claim 16 wherein each conically-shaped protruding aperture comprises (a) an interior aperture comprising an interior aperture diameter, and (b) an exterior aperture comprising an exterior aperture diameter, and wherein the interior aperture diameter is smaller than the exterior aperture diameter.

18. The system of claim 13 further comprising an anchor.

19. The system of claim 18 wherein the anchor further comprises:
a body having a first inner arm and a second inner arm; and
a crossbar having threads to receive a screw, the crossbar disposed between respective surfaces of the first inner arm and the second inner arm and coupled to sliding grooves disposed along the respective surfaces; and
an annual opening disposed on the body to receive a suture.

20. An implantable medical system comprising
an implantable medical device, wherein the implantable medical device comprises a coagulant reservoir, wherein the coagulant reservoir comprises a coagulant access port and a plurality of coagulant apertures;
an access port coupling device; and
a coagulant fluid housing wherein the coagulant fluid housing comprises coagulant fluid and wherein the coagulant fluid comprises blood plasma.

21. A porous membrane for an implantable lead, the porous membrane comprising a plurality of conically-shaped protruding coagulant apertures and a plurality of mushroom-shaped supporting members.
